SEQUENCE LISTING (28 sequences)

SEQ ID NO: 1 - DNA, 17 bp, Artificial
  example hairpin yfhC substrate
cncggcnacgaaccgag

SEQ ID NO: 2 - DNA, 15 bp, Artificial
  example hairpin yfhC substrate
ncggcnacgaaccga

SEQ ID NO: 3 - DNA, 13 bp, Artificial
  example hairpin yfhC substrate
cggcnacgaaccg

SEQ ID NO: 4 - DNA, 11 bp, Artificial
  example hairpin yfhC substrate
ggcnacgaacc

SEQ ID NO: 5 - DNA, 9 bp, Artificial
  example hairpin yfhC substrate
gcnacgaac

SEQ ID NO: 6 - DNA, 498 bp, Escherichia coli
ttgtctgaagtcgaatttagccacgaatactggatgcgtcacgcgctgacgctggcgaaacgtgcctgggatgaggggaagtgccggtcggcgcggtattagtgcataacaatcgggtaatcggcgaaggctggaaccgcccgattggtgccatgatcccaccgcacatgcagaaatcatggccctgcggcagggtggtctggtgatgcaaaattatcgtctgtcgacgccacgttgtatgtcacgcttgaaccatgtgtaatgtgtgccggagcgatgatccacagtcgcattggtgcgtggtctttggtgcgcgtgacgcgaaaactggcgctgcgggatctttaatggatgtgctgcatcatccgggttgaatcaccgagtggaaattacggaaggaatactggcggatgagtgcgcggcgttgctcagtgacttctttcgctgcgccgccaggaaattaaagcgcagaaaaaagcgcaatcctcgacggattaa

SEQ ID NO: 7 - protein, 166 aa, Escherichia coli
MSEVEFSHEYWMRHALTLAKRAWDEREVPVGAVLVHNNRVIGEGWNRPIGRHPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 8 - protein, 178 aa, Escherichia coli
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 9 - DNA, 537 bp, Escherichia coli
atgcgccgcgcttttataaccggagttttctttttgtctgaagtcgaatttagccacgaatactggatgcgtcacgcgctgacgctggcgaaacgtgcctgggatgagcgggaagtgccggtcggcgcggtattagtgcataacaatcgggtaatcggcgaaggctggaaccgcccgattggtcgccatgatcccaccgcacatgcagaaatcatggccctgcggcagggtggtctggtgatgcaaaattatcgtctgatcgacgccacgttgtatgtcacgcttgaaccatgtgtaatgtgtgccggagcgatgatccacagtcgcattggtcgcgtggtctttggtgcgcgtgacgcgaaaactggcgctgcgggatctttaatggatgtgctgcatcatccgggtatgaatcaccgagtggaaattacggaaggaatactggcggatgagtgcgcggcgttgctcagtgacttctttcgcatgcgccgccaggaaattaaagcgcagaaaaaagcgcaatcctcgacggattaa

SEQ ID NO: 10 - protein, 178 aa, Escherichia coli; identical to SEQ ID NO: 8

SEQ ID NO: 11 - DNA, 77 bp, Escherichia coli
gcatccgtagctcagctggatagagtactcggctacgaaccgagcggtcggaggttcgaatcctcccggatgcacca

SEQ ID NO: 12 - DNA, 77 bp, Yersinia pestis
gcacccatagctcagctggatagagtactcggctacgaaccgagcggtcggaagttcgaatcttcctgggtgcacca

SEQ ID NO: 13 - DNA, 77 bp, Yersinia pestis
gcacccatagctcagctggatagagtactcggctacgaactgagcggtcggaagttcgaatcttcctgggtgcacca

SEQ ID NO: 14 - DNA, 77 bp, Vibrio cholerae
gcgtccgtagctcagctggatagagtacctgtctacgaaccaggcggtcagaggttcgaatcctctcggacgcgcca

SEQ ID NO: 15 - DNA, 77 bp, Vibrio cholerae
gcgctcgtagctcagctggatagagtacctggctacgaaccaggcggtcagaggttcgaatcctctcgagcgcgcca

SEQ ID NO: 16 - DNA, 77 bp, Haemophilus influenzae
gcacccgtagctcagctggatagagtactcggctacgaaccgagcggtcagaggttcgaatcctctcgggtgcgcca

SEQ ID NO: 17 - DNA, 77 bp, Haemophilus influenzae
gcacccgtagctcagctggatagagtactcggctacgaaccgagcggtcaaaggttcgaatcctttcgggtgcgcca

SEQ ID NO: 18 - DNA, 77 bp, Pseudomonas aeruginosa
gcactcatagctcagctggatagagtactcggctacgaaccgagcggtcggaggttcgaatcctcctgagtgcgcca

SEQ ID NO: 19 - DNA, 74 bp, Pseudomonas aeruginosa
ggttcgatagctcagcctggtagagcaaccatccacgaaatggtctgtcgcgggttcgactcccgctcgaacgt

SEQ ID NO: 20 - DNA, 77 bp, Neisseria meningitidis
gcacccgtagctcagttggatagagtatctggctacgaaccagagggtcgggcgttcgaatcgctccgggtgcgcca

SEQ ID NO: 21 - DNA, 74 bp, Chlamydia pneumoniae
gcaccagtagctcagtcggatagagtacctggctacgaaccaggtggtcagaggttcgagtcctctctggtgcg

SEQ ID NO: 22 - DNA, 73 bp, Chlamydia trachomatis
gcaccagtagctcagtggatagagtacctggctacgaaccaggtggtcagaggttcaaatcctctttggtgcg

SEQ ID NO: 23 - DNA, 73 bp, Mycobacterium tuberculosis
gcgcccgtagctcaacggatagagcatctgactacggatcagaaggttgggagttcgaatctcttcgggcgcg

SEQ ID NO: 24 - DNA, 73 bp, Mycobacterium leprae
gcgcccgtagctcaacggatagagcatctgactacggatcagaaggttaggggttcgaatcccttcgggcgcg

SEQ ID NO: 25 - DNA, 77 bp, Staphylococcus aureus
gcgcccgtagctcaattggatagagcgtttgactacggatcaagaggttatgggttcgactcctatcgggcgcgcca

SEQ ID NO: 26 - DNA, 74 bp, Staphylococcus aureus
gcgcccgtagctcaattggatagagcgtttgactacggatcaagaggttatgggttcgactcctatcgggcgcg

SEQ ID NO: 27 - DNA, 77 bp, Streptococcus pyogenes
gcacccttagctcaactggatagagtacctgactacgaatcaggcggttagaggttcgactcctctagggtgcatca

SEQ ID NO: 28 - DNA, 74 bp, Streptococcus pneumoniae
gcacccttagctcaactggatagagtacctgactacgaatcaggcggttagaggttcgactcctctagggtgca

(19) United States
(12) Patent Application Publication
(10) Pub. No.: US 2005/0113296 A1
(43) Pub. Date: May 26, 2005

(54) Methods for identifying antimicrobial agents, the agents identified therewith and methods of using same

(76) Inventors: Mike G. Pollard, Alameda, CA (US); Adam Cota, Berkeley, CA (US); Corey Hoeppner, Dublin, CA (US); Ingrid E. Mehlhorn, San Francisco, CA (US); Timothy David Cole, Concord, CA (US); Joshua Alan Neiman, Albany, CA (US); T. Guy Roberts, Oakland, CA (US); Wayne Mitchell, San Francisco, CA (US)

Correspondence Address: QUINE INTELLECTUAL PROPERTY LAW GROUP, P.C., P O BOX 458, ALAMEDA, CA, 94501, US

(21) Appl. No.: 10/606,406

(22) Filed: Jun. 25, 2003

Related U.S. Application Data
(63) Continuation-in-part of application No. 10/183,923, filed on Jun. 25, 2002, now abandoned.
(63) Continuation-in-part of application No. 10/184,503, filed on Jun. 26, 2002.
(60) Provisional application No. 60/301,274, filed on Jun. 26, 2001.
(60) Provisional application No. 60/396,535, filed on Jul. 15, 2002.

(51) Int. Cl. C12Q 1/68; G01N 33/554; G01N 33/569; A61K 38/17; A61K 48/00

(52) U.S. Cl. 514/12; 435/6; 435/7.32; 514/44; 536/23.1; 424/190.1

(57) ABSTRACT

The invention provides methods of identifying compounds that inhibit specific tRNA:34A deaminases encoded by yfhC genes, compounds that inhibit such deaminases and methods of using the deaminases in a variety of in vitro and in vivo contexts, such as in the treatment and prevention of bacterial infections.

### 1. CROSS-REFERENCES TO RELATED APPLICATIONS

This application is a continuation-in-part of and claims benefit under 35 U.S.C. § 120 of application Ser. No. 10/183,923, filed Jun. 25, 2002 and application Ser. No. 10/184,503, filed Jun. 26, 2002, both of which claim benefit under 35 U.S.C. § 119(e) to provisional application Ser. No. 60/301,274 filed Jun. 26, 2001, now expired. This application also claims the benefit under 35 U.S.C. § 119(e) to provisional application Ser. No. 60/396,535, filed Jul. 15, 2002. The contents of these applications are incorporated herein by reference.

### 2. FIELD OF THE INVENTION

The present invention relates to methods for identifying antimicrobial compounds, the compounds identified thereby, and methods of using the compounds in a variety of contexts. In particular, the present invention relates to methods for identifying compounds that inhibit the reaction catalyzed by the essential product of a bacterial yfhC gene, the inhibitory compounds identified thereby and methods of using the inhibitory agents in a variety of contexts.

### 3. BACKGROUND OF THE INVENTION

Bacteria are the causative agents of numerous serious ailments, which are roughly categorized into two classes: nosocomial (hospital-acquired) infections and community-acquired infections. Owing in part to overuse and/or misuse, many strains of bacteria have become resistant to the antibiotics used to treat such infections. In fact, many observers believe the degree of resistance is so great the world is suffering from an “antibiotic crisis.” Despite the increased incidence of drug resistance, the search to find novel drug targets and novel antibiotics to combat the range of disease-causing organisms has been de-emphasized in the past forty years. As a result, there are few antibiotics that can be used with a high degree of confidence in treatment regimens. Thus, there is an urgent need for the identification of new antimicrobial agents that can be used to combat microbial infections.

### 4. SUMMARY OF THE INVENTION

The process of transfer RNA (tRNA) maturation involves the trimming of sequences from the 3′ and 5′ ends, removal of introns in eukaryotes, placement of modifications at various positions of the tRNA sequence and, finally, amino-acylation prior to productive entry into the A site of a ribosome. Among the positions commonly modified in tRNAs of all organisms is nucleotide position 34, corresponding to the “wobble” position in the anticodon. Modifications at this site increase or restrict the ability of tRNAs to recognize codons through non-Watson-Crick base pairing. Several tRNAs contain inosine at the wobble position of their anticodon, which is thought to enlarge the codon recognition capacity during protein synthesis. For example, an inosine at wobble position 34 provides for the recognition of codons ending in nucleotides U, C, or A. The modification that yields inosine in tRNA typically involves the deamination of adenosine following synthesis of the primary transcript.

It has been discovered by the present inventors that bacterial yfhC genes, which are essential to bacterial viability, encode essential enzymes that specifically deaminate an adenosine at the wobble position of a specific bacterial tRNA. This significant discovery provides the availability of therapeutic antisense oligonucleotides that interfere with the bacteria's ability to make this essential yfhC polypeptide and also enables a variety of screening assays useful for identifying compounds having therapeutic and other uses.

Thus, in one aspect the invention provides antisense oligonucleotides useful for inhibiting the ability of bacterial cells to produce an essential yfhC polypeptide. In general, the antisense oligonucleotides comprise from 8 to 30 nucleotides and include a region that hybridizes to a portion of a polynucleotide, such as a gene or mRNA, encoding a yfhC polypeptide. Such oligonucleotides will typically be single stranded, and may include modified interlinkages and/or nucleobases. When administered to bacterial cells, such antisense oligonucleotides bind to the yfhC gene or mRNA of the bacterium, interfering with the ability of the bacterium to produce the essential yfhC polypeptide encoded thereby.

In another aspect, the invention provides methods of identifying inhibitors of bacterial yfhC polypeptides. In one embodiment, the methods comprise contacting a yfhC polypeptide with a candidate compound of interest and determining whether the compound binds the yfhC polypeptide, where binding identifies the compound as an inhibitor of the yfhC polypeptide. In another embodiment the methods comprise contacting a yfhC polypeptide and a yfhC substrate with candidate compound of interest and detecting the presence or absence of a modified yfhC substrate. In one embodiment, the presence or absence of the modified substrate is detected by quantifying the amount of modified substrate produced. Compounds which reduce the amount of modified substrate produced as compared to a control reaction are identified as inhibitors of the yfhC polypeptide. As the deamination reaction catalyzed by the yfhC polypeptide is essential to bacterial viability, such inhibitory compounds can be used in a variety of in vitro and in vivo contexts to inhibit the growth or replication of, or kill altogether, bacteria expressing a yfhC polypeptide. In one embodiment, such inhibitory compounds can be used to inhibit the growth or replication of bacteria as a therapeutic approach towards the treatment or prevention of bacterial infections.

In still another aspect, the invention provides methods of identifying antimicrobial compounds. The methods generally comprise determining whether a candidate compound of interest binds or inhibits a yfhC polypeptide. In one embodiment, such antimicrobial compounds are identified by contacting a candidate compound with a yfhC polypeptide and determining whether the compound binds the yfhC polypeptide, as described above. In another embodiment, such antimicrobial compounds are identified by contacting a yfhC polypeptide and a yfhC substrate with a candidate compound of interest and detecting the presence or absence of a modified yfhC substrate, as described above. Antimicrobial compounds identified by the methods of the invention may be bactericidal or bacteristatic, and may be used in a variety of in vitro and in vivo contexts to inhibit the growth or replication of or kill bacteria. In a particular embodiment, compounds identified by the methods can be administered to humans and animals to treat or prevent bacterial infections.

In yet another aspect, the invention provides methods of identifying compounds useful for treating or preventing bacterial infections in animals and humans. The methods generally comprise determining whether a candidate compound of interest binds or inhibits a yfhC polypeptide, where the ability of the candidate compound to bind or inhibit the yfhC polypeptide identifies the compound as being useful to treat or prevent bacterial infections.

In still another aspect, the invention provides compounds identified by the various screening methods of the invention.

In still another aspect, the invention provides pharmaceutical compositions. The compositions generally comprise an antisense oligonucleotide of the invention, a polynucleotide capable of transcribing such an antisense oligonucleotide or a compound identified by the methods of the invention and a pharmaceutically-acceptable carrier, excipient or diluent. Such compositions are useful in the various therapeutic and/or prophylactic methods of the invention.

In yet another embodiment, the invention provides methods of inhibiting a yfhC polypeptide. The methods generally involve contacting a yfhC polypeptide with an amount of a yfhC inhibitory compound effective to inhibit the yfhC polypeptide. The method may be practiced in in vitro contexts or in vivo contexts.

In yet another embodiment, the invention provides a method of inhibiting the growth or replication of a bacterium. In one embodiment, the method involves administering to the bacterium an amount of an antisense oligonucleotide of the invention, or a polynucleotide capable of transcribing such an antisense oligonucleotide, effective to inhibit the production of a yfhC polypeptide, thereby inhibiting the growth of the bacterium. In another embodiment, the method involves contacting the bacterium with an amount of a yfhC inhibitory compound effective to inhibit the growth of the bacterium. The methods may be practiced in vitro contexts or in in vivo contexts.

In still another aspect, the invention provides methods of treating or preventing bacterial infections in animals and humans. The methods generally comprise administering to an animal or human subject an amount of a yfhC inhibitory compound effective to treat or prevent the bacterial infection. In one embodiment, the inhibitory compound administered is an antisense oligonucleotide of the invention or a polynucleotide capable of transcribing the same. Such inhibitory compounds interfere with the ability of the bacteria to grow or replicate in the host. In another embodiment, the inhibitory compound administered is a compound identified by a screening method of the invention. The methods may be practiced therapeutically in subjects suffering from bacterial infections or prophylactically in subjects at risk of developing bacterial infections.

In a final aspect, the invention provides kits for carrying out the various methods of the invention. In one embodiment, the kit comprises a yfhC polypeptide, an isolated polynucleotide encoding a yfhC polypeptide or a cell capable of expressing a yfhC polypeptide and a yfhC substrate. The kit may further include additional components for carrying out the methods of the invention, such as, by way of example and not limitation, buffers, labels, labeling reactions, instructions teaching methods of using the kits, etc.

### BRIEF DESCRIPTION OF THE DRAWINGS

FIG. 1A provides the nucleotide sequence of the yfhC gene of E. coli K-12 (EcoGene accession no. EG11372);

FIG. 1B provides the amino acid sequence of the yfhC polypeptide encoded by the yfhC gene of E. Coli K-12 (EcoGene accession no. EG11372);

FIG. 2 provides the amino acid sequence of the yfhC polypeptide of E. coli (GenBank accession no. P30134);

FIG. 3A provides the nucleotide sequence of the yfhC gene of E. coli K-12 MG1655 (GenBank accession no. AE000342 corresponding to GenBank accession no. ACC75612);

FIG. 3B provides the amino acid sequence of the yfhC polypeptide encoded by the yfhC gene of E. coli K-12 MG1655 (GenBank accession no. ACC75612);

FIG. 4A provides a cartoon illustrating a representative tRNA;

FIG. 4B provides a cartoon illustrating a representive hairpin oligonucleotide;

FIG. 5 provides nucleotide sequences of tRNA^{arg(ACG)} of various different bacterial species;

FIG. 6 illustrates the deamination of adenosine to yield inosine;

FIG. 7 provides HPLC profiles of myriad reactions carried out with yfhC polypeptides and yhC substrates; and

FIG. 8 provides a blow-up of the region of FIG. 7 defined by the box.

### 6. DETAILED DESCRIPTION OF THE INVENTION

6.1 Abbreviations

The abbreviations used for the genetically encoded amino acids are conventional and are as follows:

| Amino Acid | Three-Letter Abbreviation | One-Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D,” the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the N→C direction in accordance with common convention.

The abbreviations used for the genetically encoding nucleosides are conventional and are as follows: adenosine (A); guanosine (G); cytidine (C); thymidine (T); and uridine (U). The abbreviations used for common non-genetically encoding nucleosides are as follows: inosine (I), dihydrouridine (D), pseudouridine (4) and base Y (Y). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When specified on an individual basis, the one-letter abbreviation is preceded by either a “d” or an “r,” where “d” indicates the nucleoside is a 2′-deoxyribonucleoside and “r” indicates the nucleoside is a ribonucleoside. For example, “dA” designates 2′-deoxyriboadenosine and “rA” designates riboadenosine. When specified on an aggregate basis, the particular nucleic acid or polynucleotide is identified as being either an RNA molecule or a DNA molecule. Nucleotides are abbreviated by adding a “p” to represent each phosphate, as well as whether the phosphates are attached to the 3′-position or the 5′-position of the sugar. Thus, 5′-nucleotides are abbreviated as “pN” and 3′-nucleotides are abbreviated as “Np,” where “N” represents a nucleoside, e.g., A, G, C, T or U. When nucleic acid sequences are presented as a string of one-letter abbreviations, the sequences are presented in the 5′→3′ direction in accordance with common convention, and the phosphates are not indicated.

6.2 Definitions

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. Thus, for example, reference to “a polypeptide” includes a combination of two or more polypeptides, or the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

The term “candidate compound” refers to a compound which is being added to an assay system to assess the effect that the compound has upon the assay system. The candidate compound can be a synthetic compound (i.e., prepared by chemical synthesis or chemical modification), or it can be a naturally-occurring compound. As used herein, a candidate compound is meant to encompass both a single compound, as well as a group, or “pool” or “library,” of compounds.

The term “gene” refers to one or more nucleic acid sequences that encode one or more gene products. The gene can be a portion of a cellular genome, an isolated DNA sequence, or a synthetically prepared or artificially manipulated sequence. The gene can be part of the cellular genome, or it can be external to the cellular genome (for example, a component of an expression vector). In addition, the gene can be one or more components of a library of sequences.

The term “gene product” refers to product encoded by one or more genes. The gene product can be a protein, an RNA molecule, or a DNA molecule.

The term “identifying” in the context of screening assays means determining whether a candidate compound unknown to possess a particular property of interest possesses the property of interest, as well as confirming that a compound thought or known to possess a particular property of interest possesses the property of interest.

6.3 The Invention

As discussed previously, deamination of adenosine in the wobble position of tRNA anticodons provides tRNAs having a wobble inosine (I₃₄) that, through non Watson-Crick base pairing, have the ability to recognize an increased number of codons. In yeast, at least seven tRNAs having an adenosine in the wobble position (tRNA:34A) undergo deamination and carry an inosine at the anticodon wobble position. In higher eukaryotes, this tRNA:A34 deamination occurs in eight cytoplasmic tRNAs (Sprinzl et al., 1998, Nucl. Acids Res. 26:148). Quite unlike these higher microorganisms, the only tRNA substrate that undergoes an adenosine to inosine deamination reaction in eubacteria (prokaryotes), such as, for example, E. coli, is tRNA^{Arg2} (id.). The anticodon sequence of tRNA^{Arg2} is ACG.

Anticodon wobble tRNA:34A deaminations in yeast are catalyzed by an enzyme that includes two subunits: Tad2p and Tad3p (Gerber and Keller, 1999, Science 286:1146-1149). While the yeast tRNA:34A deaminase can modify bacterial tRNA^{Arg2} in addition to yeast tRNAs containing an A₃₄, the bacterial tRNA:34A deaminase is more specific. E. coli extracts containing tRNA:34A deaminase activity cannot modify any of the yeast tRNAs including an adenosine at the wobble position (Gerber and Keller, 1999, supra; Auxilien et al., 1996, J. Mol. Biol. 262:437).

The present inventors have discovered that the E. coli gene termed “yfhC” (according to systemic nomenclature of Blattner et al., 1997, Science, 277:1453-74), encodes an enzyme (yfhC polypeptide) with specific tRNA:34A deaminase activity. Like the deaminase activity observed with E. coli extracts, the deaminase activity of this E. coli yfhC polypeptide specifically recognizes ACG anticodons. Moreover, the inventors have established that the yfhC gene, and hence the encoded yfhC polypeptide, is essential for E. coli survival.

The ACG anticodon-specific tRNA:34A deaminase activity of the E. coli yfhC polypeptide was demonstrated through several steps. First, a bioinformatics approach was used to identify a set of candidate genes that have a phylogenetic distribution similar to that of tRNA^{Arg2(ACG)}, the putative substrate of the deaminase. In 60 out of the 61 bacterial genomes examined, the occurrence of this putative tRNA substrate correlates with the presence of a yfhC homolog. Second, significant homology exists between the yeast Tad2p and Tad3p sequences (Gerber and Keller 1999 supra) and three genes in E. coli, namely, yfhC, ribD (riboflavin deaminase), and the more distant cdd (deoxycytidine deaminase). The proteins encoded by these three genes share three conserved residues which coordinate a zinc atom, and are part of a larger protein motif which serves to facilitate nucleotide-zinc binding, and is believed to be involved in catalyzing the deaminase reaction. Among these three bacterial genes, yfhC was deemed the most likely candidate for the tRNA^{Arg2(ACG)} adenosine deaminase.

The candidate genes were then screened for activity using the assay disclosed in WO 01/62981 by Roberts et al. entitled “Platform for the Discovery of the Bacterial Genes Involved in RNA Modification,” filed Feb. 23, 2001. These screens showed that lysates of bacterial strains engineered to over express the E. coli yfhC gene, as well as isolates of the encoded protein (purified from the lysate through an affinity tag), were able to stimulate production of inosine in the presence of the appropriate tRNA^{Arg2} substrate by 15-30 fold over background (as measured by HPLC with a hydrolysate of the reaction mixture), confirming that the yfhC gene encodes the bacterial tRNA:34A deaminase.

To demonstrate that it is essential to bacterial viability, the E. coli yfhC gene (EcoGene EG11372) was repeatedly unsuccessfully replaced with the kanamycin resistance gene using lambda RED mediated homologous recombination as described by Datsenko and Wanner, 2000, Proc. Natl. Acad. Sci. USA 97(12):6640-6645. To further confirm that it is essential, a rescue copy of this yfhC gene was inserted into the bacterial chromosome by homologous recombination such that it was under the control of a regulatable promoter which could be shut off. The yfhC gene copy was recombined into the galK gene under the negative selection of the bactericidal compound deoxygalactose. The rescue copy of yfhC was then induced with galactose, allowing the wild type copy of the yfhC gene to be replaced with the kanamycin resistance gene. Upon removal of galactose, expression of the rescue copy of the yfhC gene was repressed and bacterial growth was suppressed, demonstrating that the gene is essential for viability.

The demonstrated specificity and importance of this E. coli yfhC polypeptide make it, as well as polynucleotides encoding the yfhC polypeptide (e.g., the yfhC gene or mRNA transcribed there from), ideal targets for therapeutic applications. For example, bacterial polynucleotides encoding the yfhC polypeptide are ideal targets for antisense oligonucleotides. Such oligonucleotides can bind to a region of the encoding polynucleotide, thereby disrupting the expression and production of the encoded yfhC polypeptide, as is well-known in the art. Since the yfhC polypeptide is essential for bacterial viability, disrupting its expression will result in the death of the bacterium.

As another example, the yfhC polypeptide is an ideal target for screening assays to identify inhibitory compounds having antibacterial properties. Since the yfhC polypeptide has limited homology to human sequences, such identified compounds will likely not inhibit the human enzyme, making the identified compounds ideal candidates for systemic administration to animals and humans as a therapeutic approach to treat or prevent bacterial infections.

6.3.1 yfhC Genes and Polypeptides

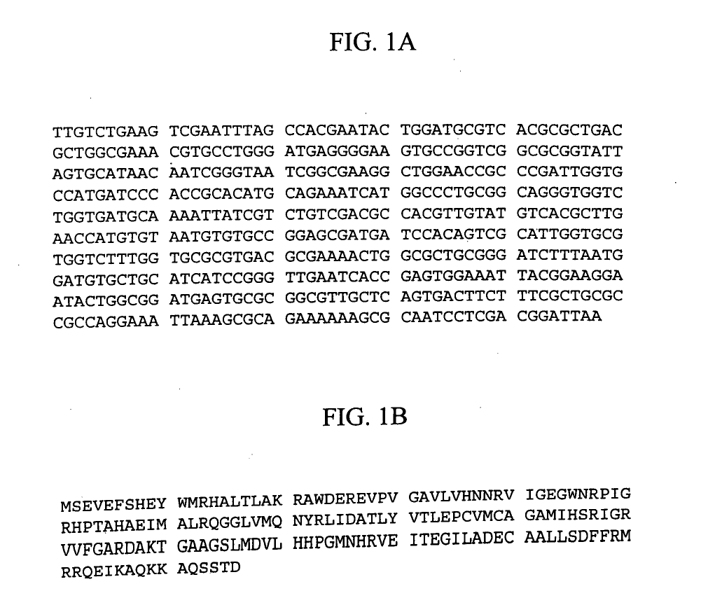

Although the various inventions described herein are exemplified with the E. coli yfhC gene and yfhC polypeptide, skilled artisans will recognize that the various methods and kits described herein are not limited to the E. coli gene and polypeptide. Indeed, homologs of the E. coli yfhC gene (Genbank accession number P30134, g267486) and polypeptide described herein occur throughout the bacterial Kingdom, although they are absent from the Mycoplasmas, Ureaplasma, Campylobacter, Helicobacter, Thermotoga, Spirochetes and members of the Archaea surveyed. Bacterial homologs to the E. coli yfhC gene are herein defined as those genes in any bacterial species which yield an E-value of less than or equal to about 10⁻²⁰ when referenced to the E. coli yfhC gene of EcoGene accession number EG11372 (FIG. 1A; see also, Blattner et al, 1997, Science 277:1453-1474 and Paulsen et al., 1992, Mol. Microbiol. 6:895-905) through application of the similarity comparison algorithm “NCBI BLAST version 2.” The BLAST algorithms are described in Zhang et al., 1998, Nucl. Acids Res. 26:3986-3990; Altschul et al., 1997, Nucl. Acids. Res. 25:3389-3402; Karlin and Altschul, 1993, Proc. Natl. Acad. Sci. USA 90:5873-5877 and Karlin and Altschul, 1990, Proc. Natl. Acad. Sci. USA 87:2264-2268. Examples of specific bacterial homologs, along with their reported E values, are provided in TABLE 1 (in TABLE 1, for comparison, the E-values for human and mouse are also provided).

As will be appreciated by skilled artisans, all of these various bacterial homologs can be used in the methods and kits described herein. Thus, unless specified otherwise, as used herein the expressions “yfhC gene” and “yfhC polypeptide” include the various bacterial homologs of the E. coli gene and the polypeptides encoded thereby. The GENBANK accession nos. of specific exemplary yfhC polypeptides that can be used in the various assays and kits defined herein are provided in TABLE 1.

TABLE 1
BLAST scores as compared to the E. coli yhfc gene of (FIG. 1A)

| | | GENBANK |
|---|---|---|
| | | yfhC polypeptide |
| Organism | E-value | Accession No. |
| E. coli O157:H7 | 10⁻⁹⁴ | gi 15832679 |
| S. typhimurium LT2 | 10⁻⁸⁵ | |
| K. pneumoniae | | |
| Y. pestis | 10⁻⁶⁵ | gi 16123110 |
| V. cholerae | 10⁻⁴³ | gi 15640880 |
| H. influenza Rd. | 10⁻⁴³ | |
| H. influenza Td | | gi 1175980 |
| L. pneumophila | 10⁻³⁸ | |
| P. aeruginosa | 10⁻³⁶ | gi 15598962 |
| N. gonorrhoeae | 10⁻³¹ | |
| N. meningitidis MC58 | 10⁻³⁰ | gi 15676827 |
| R. prowazekii | 10⁻²² | |
| H. pylori J99 | | |
| C. pneumoniae AR39 | 10⁻²⁶ | gi 16752024 |
| B. burgdorferi | | |
| T. pallidum | | |
| M. tuberculosis CDC1551 | 10⁻²⁷ | gi 158433272 |
| M. leprae | 10⁻²⁵ | gi 15828339 |
| M. avium | 10⁻²⁸ | |
| C. trachomatis | | gi 15605579 |
| M. genitalium | | |
| C. difficile | 10⁻²⁴ | |
| B. subtilis | 10⁻³² | |
| B. anthracis | 10⁻³² | |
| L. monocytogenes EGD-e | 10⁻²⁷ | |
| S. aureus ssp. aureus COL | 10⁻²⁶ | |
| S. aureus ssp. Aureus Mu50 | | gi 15923548 |
| S. epidermidis | 10⁻³⁴ | |
| E. faecalis | 10⁻³¹ | |
| E. faecium | 10⁻³⁵ | |
| S. pyogenes M1 GAS | 10⁻²⁹ | |
| S. pyogenes MGAS8232 | | gi 19745357 |
| S. pneumoniae TIGR4 | 10⁻³⁴ | gi 15899968 |
| Human | 10⁻¹⁹ | |
| Mouse | 10⁻¹⁷ | |

Mutants and/or fragments of a yfhC polypeptide may also be used in the assays and kits of the invention. The only requirement is that the yfhC polypeptide mutant or fragment specifically deaminate a yfhC substrate (described in more detail in a later section). Suitable fragments include yfhC polypeptides that are truncated at the N- and/or C-terminus by one or more amino acids, typically by about 1 to 10-20 amino acids, although fragments truncated by more amino acids may be used, provided the fragments catalyze the deamination reaction.

Alternatively, the yfhC polypeptide or fragment may include additional sequence that does not interfere with the deaminase activity of the molecule. Such sequences include, for example, sequences designed to aid purification of a recombinantly produced yfhC polypeptide, such as a His tag. Indeed, deaminase activity has been confirmed with an isolated yfhC polypeptide including a His tag fused to its N-terminus.

yfhC Polypeptide mutants useful in the methods and kits of the invention include conservative mutants in which one or more amino acids are replaced with other amino acids having similar physico-chemical properties, as is well-known in the art. Of course, yfhC polypeptide mutants including non-conservative substitutions may also be used, so long as the particular mutant deaminates a yfhC substrate. Thus, unless indicated otherwise, the expression “yfhC polypeptide” as used herein specifically includes such mutants and/or fragments in addition to the full-length wild-type polypeptide.

6.3.2 Methods of Making yfhC Polypeptides

yfhC Polypeptides useful in the screening methods and kits described herein may be supplied in purified or isolated form, or may be supplied in the form of cellular extracts or lysates. For example, a yfhC polypeptide may be isolated from an appropriate bacterial source, such as the various bacterial strains listed in TABLE 1, using conventional isolation techniques. The bacterial strain can express the yfhC polypeptide endogenously or it can be engineered to over-express the yfhC polypeptide. Alternatively, such cells may be lysed and the yfhC polypeptide supplied in the form of a cell lysate or extract without further isolation or purification. As another example, the yfhC polypeptides can be synthesized via conventional solid or solution phase peptide and/or protein synthesis techniques, as are well-known in the art.

As mentioned previously, the yeast tRNA:34A deaminase is a heterodimer consisting of TAD2 and TAD3 monomers (Gerber and Keller, 1999, Science 286:1146-1149). Given that there is little homology between Tad3 and yfhC, it is presumed that the yfhC polypeptide is active as a monomer or as a homodimer. However, it is expected that the various yfhC homologs described herein may form active heterodimers with any of the other yfhC homologs. Thus, whether supplied in purified form or as a cellular extract or lysate, the yfhC polypeptide may be in the form of a monomer, a homodimer or a heterodimer.

A variety of expression vectors that can be employed to engineer bacterial and/or eukaryotic cells capable of over-expressing a particular yhfc polypeptide of interest are well known in the art. Such expression vectors include, but are not limited to, viruses, plasmids, episomes, transposons, phages, artificial chromosomes (such as a bacterial or yeast artificial chromosome), and the like. Typically, the expression vector comprises regulatory sequences, including, for example, a promoter, operably linked to the polynucleotide encoding the yhfC polypeptide. Exemplary promoters for use in constructing yhfC polypeptide over-producing strains include, but are not limited to, the E. coli lac or trp promoter, SV40 promoter, phage lambda PL promoter, CMV promoter, arabinose promoter, phosphate promoter and T7 promoter. Large numbers of suitable vectors and promoters are known to those of skill in the art, and are commercially available.

Methods of introducing genetic material into cells, including plant and animal cells, e.g., for cloning and sequencing and/or for expression and selection of encoded molecules are generally available, as are methods of expressing proteins encoded by such nucleic acids. See, e.g., Sambrook et al., 1989, Molecular Cloning—A Laboratory Manual (2nd Ed.), Vol. 1-3, Cold Spring Harbor Laboratory, Cold Spring Harbor, N.Y.; Current Protocols in Molecular Biology, F.M. Ausubel et al., Eds., Current Protocols, (a joint venture between Greene Publishing Associates, Inc. and John Wiley & Sons, Inc., supplemented through 2000); Freshney, 1994, Culture of Animal Cells, a Manual of Basic Technique, 3^{rd} Edition, Wiley-Liss, New York (and the references cited therein); Humason, 1979, Animal Tissue Techniques, 4^{th} Edition, W.H. Freeman and Company, New York; and Ricciardelli et al., 1989, In Vitro Cell Dev. Biol. 25:1016-1024. References describing nucleic acid manipulation techniques are known in the art, and include, for example, Berger and Kimmel, Guide to Molecular Cloning Techniques, Methods in Enzymology volume 152 (Academic Press, Inc., San Diego, Calif.); PCR Protocols A Guide to Methods and Applications, Innis et al., Eds., 1990, Academic Press Inc., San Diego, Calif.; De Lorenzo and Timis, 1994, Methods in Enzymology 235:385-404; Kleckner et al., 1991, Methods in Enzymology 204, chapter 7; as well as Sambrook and Ausubel, both supra. These and other references cited therein describe cell culture techniques and recombinant nucleic acid methodologies that may be used to engineer cell strains that over-express yfhC polypeptides according to the invention.

6.3.3 Screening Assays

The various screening assays of the invention typically involve assessing whether the yfhC polypeptide binds a candidate compound or catalyzes its specific deaminase reaction in the presence of a candidate compound of interest. In some embodiments, random candidate compounds are screened directly in such functional assays to identify those candidate compounds that inhibit the yfhC polypeptide. In other embodiments, candidate lead compounds are first identified in simple binding assays. Those compounds which bind the yfhC polypeptide are then optionally further assayed for inhibitory activity, and hence antibacterial activity, in functional assays.

Any screening technique known in the art for determining whether compounds bind one another can be used to screen for compounds that bind a yfhC polypeptide. The compounds screened can range from small organic molecules to large polymers and biopolymers, and can include, by way of example and not limitation, small organic compounds, saccharides, carbohydrates, polysaccharides, lectins, peptides and analogs thereof, polypeptides, proteins, antibodies, oligonucleotides, polynucleotides, nucleic acids, etc. the compounds can be naturally occurring, or they can be synthetic.

In one embodiment, the candidate compounds screened are small organic molecules having a molecular weight in the range of about 100-1000 daltons. Such candidate molecules will often comprise cyclical structures composed of carbon atoms or mixtures of carbon atoms and one or more heteroatoms and/or aromatic, polyaromatic, heteroaromatic and/or polyaromatic structures. The candidate agents may include a wide variety of functional group substituents. In one embodiment, the substituent(s) are independently selected from the group of substituents known to interact with proteins, such as, for example, amine, carbonyl, hydroxyl and carboxyl groups.

In another embodiment, the compounds screened are natural products, such as cellular extracts or fractions from microorganisms or other natural sources. Such natural products typically have molecular weights in the range of about 100-2500 daltons.

The candidate compounds may be screened on a compound-by-compound basis or, alternatively, using one of the myriad library techniques commonly employed in the art. For example, synthetic combinatorial compound libraries, natural products libraries and/or peptide libraries may be screened using the assays of the invention to identify compounds that bind a yfhC polypeptide. The candidate compounds may be assessed for the ability to bind a yfhC polypeptide per se, or they may be assessed for the ability to competitively bind a yfhC polypeptide in the presence of another compound that inhibits the yfhC polypeptide (“known inhibitor”). Such known inhibitors can be identified in the various screening assays described herein. These competitive binding assays can identify compounds that bind the yfhC polypeptide at approximately the same site as the natural substrate, and can therefore be used to identify inhibitory compounds without the need for further functional screens. Myriad techniques for carrying out competitive binding assays are known in the art. Any of these techniques may be employed in the present invention.

Binding experiments may be conducted wholly in solution or, alternatively, either the yfhC polypeptide or the candidate compound may be immobilized on a solid support. For example, the yfhC polypeptide or the candidate compound may be attached to a glass or other bead or to a solid surface such as, for example, the bottom of a petri dish. The immobilization may be mediated by non-covalent interactions or by covalent interactions. Methods for immobilizing myriad types of compounds and proteins on solid supports are well-known. Any of these methods may be used to immobilize the yfhC polypeptide and/or candidate compound on solid supports. As a specific example, yfhC polypeptides including a His tag may be immobilized on a Ni²⁺ or Zn²⁺ column using art-known techniques.

The binding assays may employ a purified yfhC polypeptide or, alternatively, the assays may be carried out with lysates or extracts of cells that express the yfhC polypeptide, either endogenously or recombinantly.

Whether carried out in solution or with an immobilized yfhC polypeptide or candidate compound, the yfhC polypeptide and candidate compound are typically contacted with one another under conditions conducive to binding. Although the actual conditions used can vary, typically, the binding assays are carried out under physiological conditions. The concentrations of yfhC polypeptide and candidate compound used will depend upon, among other factors, whether the yfhC polypeptide or candidate compound is immobilized or free in solution, the binding affinities of candidate compounds, etc. Actual concentrations suitable for a particular assay will be apparent to those of skill in the art.

In many embodiments of the kits and assays of the invention it may be convenient to employ a labeled yfhC polypeptide and/or a labeled candidate compound. For example, in one convenient embodiment, binding is assessed by contacting an immobilized candidate compound with a labeled yfhC polypeptide and assaying for the presence of immobilized label. For such embodiments, the label may be a direct label, i.e., a label that itself is detectable or produces a detectable signal, or it may be an indirect label, i.e., a label that is detectable or produces a detectable signal in the presence of another compound or that induces another compound to produce a detectable signal. The method of detection will depend upon the labeled used, and will be apparent to those of skill in the art.

Examples of suitable direct labels include radiolabels; fluorophores, chromophores, chelating agents, particles, chemiluminescent agents and the like. Suitable radiolabels include, by way of example and not limitation, ³H, ¹⁴C, ³²P, ³⁵S, ³⁶Cl, ⁵⁷Co, ⁵⁸Co, ⁵⁹Fe, ⁹⁰Y, ¹²⁵I, ¹³¹I and ¹⁸⁶Re. Suitable fluorophores include, by way of example and not limitation, fluorescein, rhodamine, phycoerythrin, Texas red, free or chelated lanthanide series salts such as Eu³⁺ and the myriad fluorophores available from Molecular Probes Inc., Eugene, Oreg. Examples of suitable colored labels include, by way of example and not limitation, metallic sol particles, for example, gold sol particles such as those described by Leuvering, U.S. Pat. No. 4,313,734; dye sole particles such as these described by Gribnau et al., U.S. Pat. No. 4,373,932 and May et al. International Publication No. WO 88/08534; dyed latex particles such as those described Snyder, EP 0 280 559 and EP 0 281 327 and dyes encapsulated in liposomes, as described by Campbell et al., U.S. Pat. No. 4,703,017. Other direct labels that may be used will be apparent to those of skill in the art.

Examples of suitable indirect labels include enzymes capable of reacting with or interacting with a substrate to produce a detectable signal (such as those used in ELISA and EMIT immunoassays), ligands capable of binding a labeled moiety, and the like. Suitable enzymes useful as indirect labels include, by way of example and not limitation, alkaline phosphatase, horseradish peroxidase, lysozyme, glucose-6-phosphate dehydrogenase, lactate dehydrogenase and urease. The use of these enzymes in ELISA and EMIT immunoassays is described in detail in Engvall, 1980, Methods in Enzymology, 70:419-439 and U.S. Pat. No. 4,857,453. Suitable molecules capable of binding a labeled molecule include, by way of example and not limitation, biotin, which can bind a labeled avidin or streptavidin.

Methods of labeling proteins and compounds with a variety of labels such as those described above are well-known. Any of these methods may be used to label yfhC polypeptides and/or candidate compounds. For example, a yfhC polypeptide may be labeled with a fluorophore such as fluorescein by incubating the yfhC polypeptide with, for example, fluorescein isothiocyanate, using conventional techniques. Alternatively, a yfhC polypeptide (or a candidate compound produced by recombinant techniques) can be labeled metabolically by culturing cells that express the yfhC polypeptide in the presence of culture medium supplemented with a metabolic label, such as, by way of example and not limitation, [³⁵S]-methionine, one or more [¹⁴C]-labeled amino acids, one or more [¹⁵N]-labeled amino acids and/or one or more [³H]-labeled amino acids (with the tritium substituted at non-labile positions).

In one embodiment of the invention, candidate compounds may be screened for the ability to bind a yfhC polypeptide using an affinity chromatography technique. For example, a yfhC polypeptide may be attached to a chromatography resin according to standard techniques to create a yfhC polypeptide affinity resin and this yfhC polypeptide affinity resin used to identify compounds that bind the resin. Alternatively, the candidate compound could be bound to the resin and the resin used to determine whether it binds a yfhC polypeptide. In another alternative embodiment, a known inhibitory compound may be attached to the chromatography resin. This resin may then be used to bind a yfhC polypeptide and the bound complex used to identify compounds that compete for binding the yfhC polypeptide with the known inhibitory compound, typically by washing the resin with a candidate compound and determining whether the candidate compound disrupts the yfhC polypeptide-inhibitory compound complex by assaying for the release of yfhC polypeptide from the resin. The known inhibitory compound employed may be an inhibitory compound identified using the various screening assays described herein.

Although candidate compounds may be screened for the ability to bind a yfhC polypeptide on a compound-by-compound basis, it may be more convenient to screen large numbers of candidate compounds simultaneously using one of the many library screening methodologies known in the art. One art-known approach uses recombinant bacteriophage to produce large libraries of peptides which can then be screened in a variety of formats for binding to a yfhC polypeptide. Using such phage methods, very large libraries of candidate peptides can be constructed (e.g., 10⁶-10⁸ peptides) and screened for binding with a yfhC polypeptide. Methods for constructing and screening such “phage display” libraries are described, for example, in Scott et al., 1990, Science 249:386-390; Cwirla et al., 1990, Proc. Natl. Acad. Sci. 87:6378-6382; 1990); Devlin et al., 1990, Science 249:404-406 (1990); U.S. Pat. No. 5,427,908; U.S. Pat. No. 5,432,018; U.S. Pat. No. 5,580,717 and U.S. Pat. No. 5,723,286. Other non-limiting examples of recombinant library methodologies that may be used in connection with the assays of the invention are described in U.S. Pat. No. 6,156,571; U.S. Pat. No. 6,107,059 and U.S. Pat. No. 5,733,731.

A second art-known approach uses chemical methods to synthesize libraries of compounds, such as small organic compounds, peptides and/or peptide analogs, attached to beads or wafers that can then be conveniently screened for binding with a yfhC polypeptide. The libraries may be encoded or non-encoded. Methods of synthesizing such immobilized libraries, as well as methods of screening the libraries are described, for example, in Houghten, 1985, Proc. Natl. Acad. Sci. USA 82:5131-5735; Geysen et al., 1986, Molecular Immunology 23:709-715; Geysen et al., 1987, J. Immunologic Method 102:259-274; Frank et al., 1988, Tetrahedron 44:6031-6040; Fodor et al., 1991, Science 251:767-773; Furka et al., 1988, 4th International Congress of Biochemistry, Volume 5, Abstract FR:013; Furka, 1991, Int. J. Peptide Protein Res. 37:487-493; Frank, 1992, Tetrahedron 48:9217-9232; Needels et al., 1993, Proc. Natl. Acad. Sci. USA 90:10700-10704; DeWitt et al., 1993, Proc. Natl. Acad. Sci. USA 90:6909-6913; Frank et al., 1993, Biorg. Med. Chem. Lett. 3:425-430; Ohlmeyer et al., 1993, Proc. Natl. Acad. Sci. USA 90:10922-10926; WO 92/00252; WO 9428028; U.S. Pat. No. 6,329,143; U.S. Pat. No. 6,291,183; U.S. Pat. No. 5,885,837; U.S. Pat. No. 5,424,186; U.S. Pat. No. 5,384,261; U.S. Pat. No. 6,165,717; U.S. Pat. No. 6,143,497; U.S. Pat. No. 6,140,493; U.S. Pat. No. 5,789,162; U.S. Pat. No. 5,770,358; U.S. Pat. No. 5,708,153; U.S. Pat. No. 5,639,603; U.S. Pat. No. 5,541,061; U.S. Pat. No. 5,525,735; U.S. Pat. No. 5,525,734; U.S. Pat. No. 6,261,776; U.S. Pat. No. 6,239,273; U.S. Pat. No. 5,846,839; U.S. Pat. No. 5,770,455; U.S. Pat. No. 5,770,157; U.S. Pat. No. 5,609,826; U.S. Pat. No. 6,001,579; U.S. Pat. No. 5,968,736; U.S. Pat. No. 5,962,337; U.S. Pat. No. 5,789,172; U.S. Pat. No. 5,721,099; U.S. Pat. No. 5,565,324; U.S. Pat. No. 5,010,175; and U.S. Pat. No. 4,631,211. For reviews of some of these techniques, see Ellman et al., 1996, Account, Chem. Res. 29:132-143; Gallop et al., 1994, J. Med. Chem. 37:1233-1251; Gordon et al., 1994, J. Med. Chem. 37:1385-1401. Non-limiting examples of solid-phase chemical synthesis strategies and conditions useful for synthesizing combinatorial libraries of small organic and other compounds may be found in Bunin, 1998, The Combinatorial Index, Academic Press, London, England (see, e.g., Chapter 1-5) and Hermkens et al., 1996, Tetrahedron 52:4527-4554, as well as the references cited therein.

Another art-known approach utilizes solution-phase chemical synthesis techniques to synthesize libraries of compounds, such as, for example, libraries of small organic compounds, which may then be screened in the assays of the invention. Methods for synthesizing and screening such solution-phase libraries are well-known and are described, for example, in Bunin, 1998, The Combinatorial Index, Academic Press, England (see, e.g., Chapter 6); International Publication No. WO 95/02566; U.S. Pat. No. 5,962,736; U.S. Pat. No. 5,766,481; U.S. Pat. No. 5,736,412 and U.S. Pat. No. 5,712,171, and the references cited therein.

Yet another art-known approach that may be used utilizes complementary libraries (see, WO 98/19162 to Fowlkes et al.). In this approach, a first, “potential surrogate” library of compounds is first screened to identify a “surrogate ligand” that binds a targets (in this case a yfhC polypeptide). A second library of compounds is then screened in the presence of the surrogate ligand to identify compounds which inhibit binding between the target and surrogate ligand.

Additional review articles, references, patents and books describing myriad techniques for synthesizing and screening libraries of compounds for the ability to bind another compound such as a yfhC polypeptide can be found at Lebl & Leblova: Dynamic Database of References in Molecular Diversity, Internet http://www.5z.com (see especially the diversity information pages at http://www.5z.com/divinfo).

Once a candidate compound that binds the yfhC polypeptide has been identified, further assays may be carried out to characterize the binding characteristics of the compound, for example, to determine its binding affinity, dissociation constant (Kd), on- and/or off-rates, etc., using well-known techniques. For example, binding affinities can be determined using saturation kinetics and Scatchard analysis. For saturation kinetics, the binding assay can be performed with increasing concentrations of the candidate compound, which is typically labeled with, for example, a radiolabel. Competitive binding experiments with a known inhibitory compound, for example an inhibitor identified using the functional assays described herein, can be carried out with increasing concentrations of unlabeled candidate compound and a fixed concentration of labeled (for example radiolabled) inhibitory compound. Methods for carrying out such saturation kinetics and competitive binding assays that may be used to characterize the binding characteristics of the candidate compound are well-known in the art.

An alternative method for characterizing binding characteristics of a plurality of compounds in parallel that may be used in connection with the invention is described in U.S. Pat. No. 5,324,633.

In one embodiment of the invention, the candidate compounds identified will have a dissociation constant (K_{d}) on the order of 1 mM, 100 μM, 10 μM, 1 μM, 100 nM, 10 nM, 1 nM or even lower. In another embodiment of the invention, the candidate compounds identified will exhibit an IC₅₀ in a competitive binding assay with a known inhibitory compound on the order of 1 mM, 100 μM, 10 μM, 1 μM, 100 nM, 10 nM, 1 nM or even lower. In this context, the IC₅₀ represents the concentration of candidate compound that displaces 50% of the bound inhibitory compound. Suitable assays for measuring such IC₅₀s are well-known.

The ability of candidate compounds that bind the yfhC polypeptide to inhibit the polypeptide's ability to catalyze the specific tRNA:34A deamination reaction may be confirmed using functional assays. Alternatively, these functional assays may be used as primary screens to identify compounds having yfhC inhibitory, and thus antibacterial, activity without first screening for binding. In such functional assays, a yfhC polypeptide and a yfhC substrate are contacted with a candidate compound and the presence or absence of a modified yfhC substrate is detected. The presence or absence of the modified substrate, the amount of modified substrate relative to unmodified substrate or the amount of modified substrate relative to that produced by a control reaction provides information about whether the compound inhibits the yfhC polypeptide. For example, compounds which reduce by at least about ≧50% the ability of the yfhC polypeptide to modify the substrate as compared to a control reaction are identified as inhibitory compounds. Compounds that exhibit higher degrees of inhibition, for example on the order of ≧60%, ≧65%, ≧70%, ≧75%, ≧80%, ≧85%, ≧90%, ≧95%, or even higher degrees of inhibition, are particularly useful.

The functional screens may be carried out with an isolated yfhC polypeptide or, alternatively, with lysates or extracts of cells that express, either endogenously or recombinantly, the yfhC polypeptide, as described above. The various yfhC substrates that can be used are described in some detail, below.

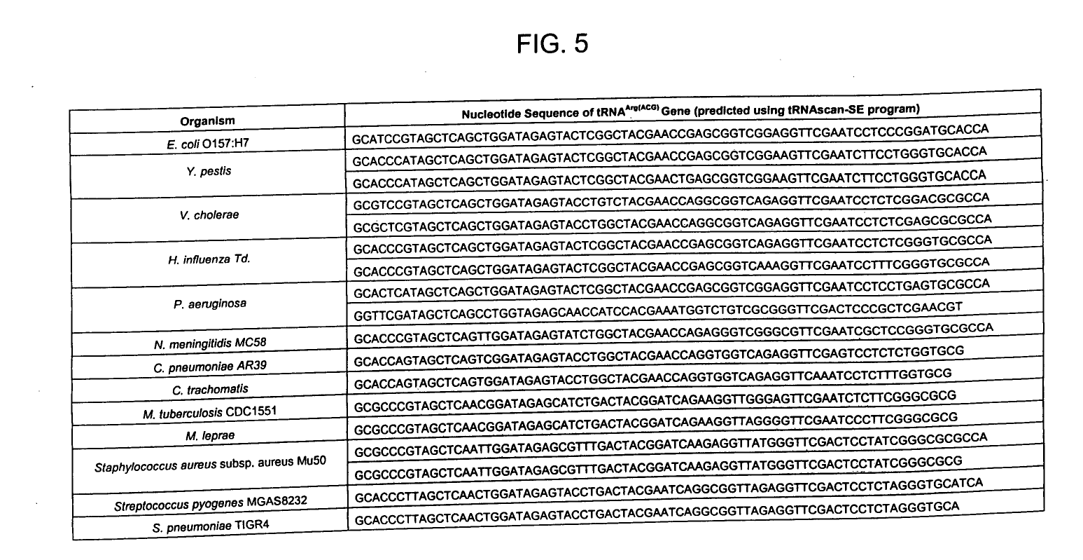

As discussed previously, the yfhC polypeptides described herein are tRNA:34A deaminases that specifically recognize bacterial tRNAs having a ACG anticodon sequence. These yfhC polypeptides do not deaminate other tRNAs having an anticodon wobble A₃₄. For example, yeast tRNAs having an anticodon wobble A are not deaminated by yfhC polypeptides. Accordingly, one yfhC substrate useful in the methods and kits of the invention is a tRNA which includes an ACG anticodon. Such tRNA^{Arg(ACG)} substrates are well-known, and include, by way of example, and not limitation, tRNA^{Arg(ACG)} from bacteria such as E. coli and the various other bacterial species having a yfhC gene, such as the bacterial strains listed in TABLE 1 having an E-value of ≦10⁻²⁰. Non-limiting examples of nucleotide sequences of specific bacterial tRNA^{Arg(ACG)}s that can be used as yfhC substrates in the assays and kits of the invention are provided in FIG. 5.

Quite surprisingly, it has been discovered that the entire tRNA^{Arg(ACG)} molecule is not required for catalytic activity. Indeed, the E. coli yfhC polypeptide of FIG. 1A has been shown to catalyze deamination of a highly truncated hairpin RNA derived from the anticodon stem loop of E. coli tRNA^{Arg2}. This is quite unlike the yeast tRNA:34A deaminase (TAD2/TAD3 heterodimer), which depends upon the global, three-dimensional structure of its entire tRNA substrate for activity (Auxilian et al., 1996, J. Mol. Biol. 262:437-458.

Thus, also useful as yfhC substrates in the methods and kits of the invention are oligonucleotides that include the sequence ACG. Such oligonucleotides may be composed wholly of gene-encoding nucleosides or, alternatively may include one or more non-encoding nucleosides or modified nucleosides, and may comprise from 3 nucleotides up to about 80 nucleotides, or even more. The oligonucleotides may be composed wholly of native 2′-deoxyribophosphate (DNA) interlinkages, wholly of native ribophosphate (RNA) interlinkages, or may include mixtures of such interlinkages. The oligonucleotides may be linear or, alternatively, they may adopt secondary or tertiary structures. For example, the oligonucleotide may adopt a hairpin or other secondary structure.

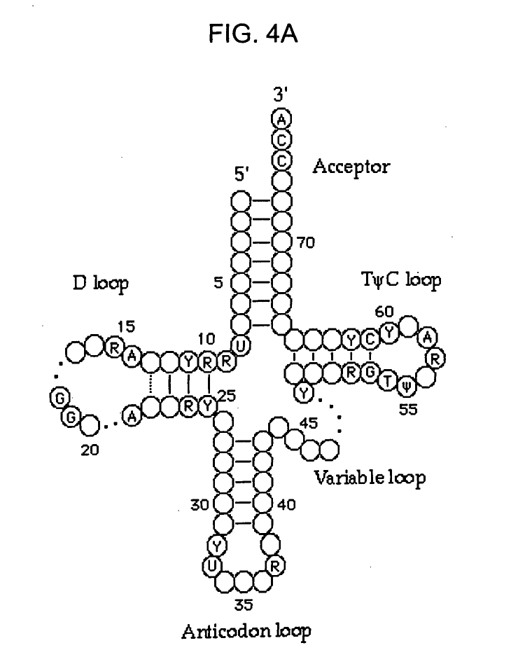

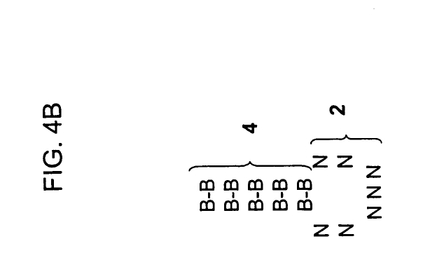

In embodiments including more than about 6-7 nucleotides, it is preferred that the oligonucleotide include secondary, and optionally tertiary, structural features of a tRNA. The nucleotide sequence may correspond to the sequence of a naturally-occurring tRNA, or it may be a completely synthetic sequence. For example, referring to FIG. 4A, the yfhC substrate may be an oligonucleotide having a nucleotide sequence capable of adopting the overall global structure of the illustrated tRNA, provided that it includes an ACG anticodon sequence. In some embodiments, the remainder of the sequence may correspond to that of a naturally occurring tRNA, while in other embodiments it may not.

In another embodiment, the yfhC substrate is a truncated TRNA^{Arg(ACG)}. The substrate may be truncated at either or both of the 5′ and 3′ ends by any number of nucleotides, provided that the resultant substrate includes the anticodon loop.

Referring to FIG. 4A, a significant structural feature conserved in all tRNAs is the hairpin including the anticodon loop (anticodon stem loop), which includes (approximately) nucleotides #26-43. In FIG. 4A, nucleotides conserved in all tRNAs are identified with letters, all others are shown as circles. Thus, in another embodiment, the yfhC substrate is a hairpin oligonucleotide that includes an ACG sequence in its loop region. Referring to FIG. 4B, a hairpin comprises a loop region 2 and a stem region 4. The loop region may include from 2 to about 10 nucleotides, but preferably includes 7 nucleotides of the sequence NNACGNN, where N represents any base, as this is the geometry of the anticodon loop of a tRNA. The stem region 4 can comprise from one to about 10 base pairs, but is preferably from 3 to 5 base pairs in length. The stem may include overhangs (5′ or 3′), or may be blunt-ended. In one embodiment, a hairpin loop yfhC substrate is an oligonucleotide of the formula:
B₍₁₋₅₎NNACGNNB₍₁₋₅₎

where each N independently represents any base and each B independently represents a base that forms Watson-Crick base pairs with a corresponding B. Preferred hairpin substrates are those having a nucleotide sequence that corresponds to the sequence of all or a portion of the anticodon loop of a bacterial tRNA^{Arg(ACG)}, such as the various tRNA^{Arg(ACG)}s of FIG. 5. Non-limiting examples of specific hairpin yfhC substrates that can be used in the methods and kits of the invention include the following oligonucleotides (the anticodon ACG is underlined):

| | |
|---|---|
| C U/T C G G C U/T ACG A A C C G A G | (SEQ ID NO:1) |
| U/T C G G C U/T ACG A A C C G A | (SEQ ID NO:2) |
| C G G C U/T ACG A A C C G | (SEQ ID NO:3) |
| G G C U/T ACG A A C C | (SEQ ID NO:4) |
| G C U/T ACG A A C | (SEQ ID NO:5) |

Depending upon the nature of the yfhC substrate, it can be isolated from cells or synthesized using standard techniques of oligonucleotide synthesis. For example, tRNA^{Arg(ACG)} yfhC substrates can be isolated from a suitable bacterial strain using standard techniques. tRNAs are also available from commercial sources. Methods suitable for chemically synthesizing yfhC substrates are well-known. Reagents and kits for carrying out such synthesis in an automated fashion are available commercially.

The presence or absence of a modified yfhC substrate can be monitored or assessed using a variety of means. Non-limiting examples of analytical techniques suitable for use in determining whether a yfhC substrate has been modified, the extent of modification, and/or the type of modification include, but are not limited to, mass spectrometry, thin layer chromatography (TLC), high pressure liquid chromatography (HPLC), capillary electrophoresis (CE), NMR spectroscopy, X-ray crystallography, cryo-electron microscopic analysis, or combinations thereof.

Traditionally, analysis of modifications of RNA molecules has been performed using thin layer chromatographic techniques on radioactive substrates. References exist which address this traditional analytical methodology. More recently, mass spectrometry (MS) has been used by several academic groups to assess the modification states of RNA molecules.

Mass spectrometry is a particularly versatile analytical tool, and includes techniques and/or instrumentation such as electron ionization, fast atom/ion bombardment, MALDI (matrix-assisted laser desorption/ionization), electrospray ionization, tandem mass spectrometry, and the like. A brief review of mass spectrometry techniques commonly used in biotechnology can be found, for example, in Mass Spectrometry for Biotechnology by G. Siuzdak (1996, Academic Press, San Diego).

When the reaction is monitored by mass spectrometry, the assay solutions (containing the newly modified yfhC substrate) are prepared according to known techniques, either with or without first digesting the reaction to mononucleosides (described in more detail, below), and then transferring to a suitable solvent system. Analysis by mass spectrometry yields a spectrogram from which both the mass and composition of the substrate molecule can be determined, in both the modified (e.g., a tRNA^{arg(ACG)}, etc.) or unmodified states (e.g., a tRNA^{arg(ACG)}, etc.). By direct comparison of these spectrograms, the modification state of the substrate molecule is revealed in a computationally straightforward manner. The presence or absence of modifications on these molecules determines, e.g., whether the compound is an inhibitor of the yfhC polypeptide.

Alternatively, the assay solutions containing the modified substrate molecules are prepared for NMR spectroscopy by removal of the original solvent solution (for example, by lyophilization), and re-dissolution into a suitable solvent, such as a deuterated solvent solution. Suitable deuterated solvent solutions include, but are not limited to D₂O (deuterium oxide), CDCl₃, DMSO-d₆, acetone-d6, and the like (available from Cambridge Isotope Labs, Andover, Mass.; www.isotope.com). Optionally, the samples can be analyzed using LC-NMR spectroscopy. Analysis by these methodologies can provide information related to both the presence of one or more modifications, as well as the type or identity of the modification (see, for example, NMR of Macromolecules: A Practical Approach, G. C. K. Roberts, ed., 1993, Oxford University Press, New York).

The assay reactions can also be monitored for the presence or absence of a modified yfhC substrate using HPLC. According to this embodiment, the assay reaction is first treated with a reagent to digest the yfhC substrate into nucleosides and then analyzed by HPLC. Comparison of the elution profile of the digested assay reaction with standard profiles of various nucleosides provides information about whether, and the extent to which the yfhC substrate was modified. For example, the assay reaction mixture can be monitored by HPLC for the presence of inosine (the product of yfhC polypeptide-mediated deamination). The appearance of inosine indicates the yfhC substrate has been modified. Comparison of the efficiency or extent of the yfhC reaction in the presence of a candidate compound as compared to a control reaction carried out in the absence of a test compound determines whether the compound is an inhibitor of the yfhC polypeptide. HPLC columns, buffers and elution conditions suitable for monitoring for the presence or absence of the modified yfhC substrate are provided in the Examples section.

The treatment used to digest the yfhC substrate will depend upon the identity of the substrate and will be apparent to those of skill in the art. For example, yfhC substrates that are DNAs may be digested enzymatically with DNA exonucleases. Substrates that are RNAs may be digested enzymatically with RNA exonucleases. Alternatively, the substrate may be digested chemically using well-known techniques. The digestive reaction can be, but need not be, carried out to completion.

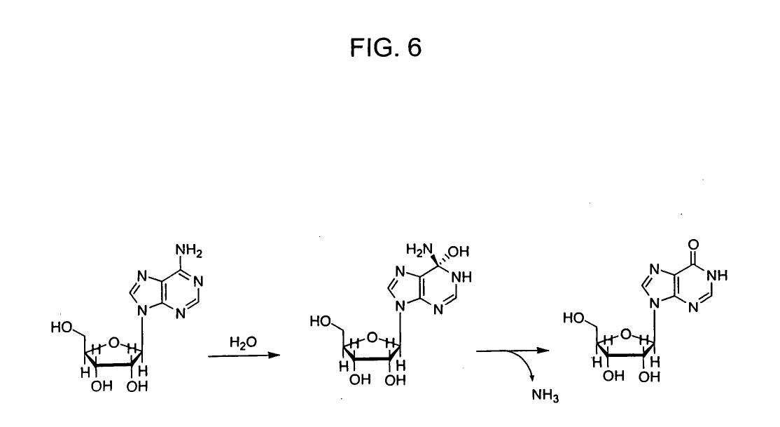

Since the deamination reaction catalyzed by the yfhC polypeptide releases ammonia or ammonium ion (the deamination reaction is illustrated in FIG. 6), the presence or absence of a modified yfhC substrate may be determined by monitoring the reaction for the release of ammonia or ammonium ion. In this embodiment, a measured release of ammonia or ammonium above a predetermined background level indicates yfhC polypeptide activity. Candidate compounds exhibiting inhibitory activity will reduce the amount of ammonia or ammonium ion generated as compared to a control reaction carried out in the absence of the candidate compound.

Typically, the assay is first carried out in the absence of the candidate compound to establish the background or baseline for the reaction components. The reaction is then run at varying concentrations of yfhC substrate such that the yfhC polypeptide is limiting and the reaction is linear with respect to signal (released ammonia or ammonium ion) as a function of time. Inhibitory candidate compounds yield a lower signal than the control at a predetermined time end point, or change the slope of a plot of signal vs. time as compared to a control reaction.

Release of ammonia or ammonium ion can be monitored using a variety of art-known techniques. A specific example is the glutamate dehydrogenase enzyme-couple assay described in van Anken et al., 1974, Clin. Chem. Acta 56:151 and Mondzac et al., 1965, J. Lab. Clin. Med. 66:526. Commercial kits for carrying out this glutamate dehydrogenase assay are commercially available (e.g., Kit #171-A available from Sigma, St. Louis, Mo.). In this assay, the ammonia or ammonium produced in the deamination reaction is used as the limiting reagent in the following reaction catalyzed by glutamate dehydrogenase:
2-oxoglutarate+NH₃+NADPH=glutamate+NADP
The reaction is monitored by following the absorbance at 340 nm attributable to accumulation of NADP.

Additional methods optionally adapted to the present invention, including screens for potential modulators using cellular assay solutions and other screens for antibiotic activity are described in detail in International Application No. PCT/US01/05920 by Roberts et al. entitled “Platform for the Discovery of the Bacterial Genes Involved in RNA Modification,” filed Feb. 23, 2001.

6.4 Antisense Oligonucleotides

As the yfhC gene is essential to the viability of the microorganism, disrupting the expression of the yfhC gene provides a means of inhibiting the growth of, or killing the target microorganism. Thus, also provided by the invention are antisense oligonucleotides that are capable of inhibiting or shutting down altogether the ability of a target microorganism having a yfhC gene to express the gene. Such antisense oligonucleotides may bind to the yfhC gene, thereby disrupting transcription of yfhC mRNA, or may bind to the mRNA to disrupt expression of the yfhC polypeptide. The principles of designing antisense oligonucleotides capable of inhibiting the expression of particular gene products are well-known. Typically, such antisense oligonucleotides hybridize with a portion or region of a polynucleotide encoding the yhfc polypeptide (e.g., yfhC gene or yfhC mRNA) such that the transcription of the gene or translation of the mRNA is disrupted. Polynucleotide sequences encoding yfhC polypeptides that can be used to construct such are antisense oligonucleotides are those described previously (see also FIGS. 1-3 and 7). In one embodiment, the antisense oligonucleotide is designed to hybridize to the wild-type yfhC gene or mRNA of a target pathogen of interest. For example, antisense oligonucleotides useful to treat or prevent E. coli infections may be designed to hybridize to the E. coli yfhC gene of FIG. 1A.

As used herein, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target.

It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

As used herein, the term “oligonucleotide” refers to an oligomer or polymer of ribonucleic acid (RNA) or 2′-deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases. Particularly preferred are antisense oligonucleotides comprising from about 8 to about 30 nucleobases (i.e., from about 8 to about 30 linked nucleosides). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al., 1991, Science 25:1497-1500.

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH—, —CH₂—N(CH₃)—N(CH₃)—CH₂— and —O—N(CH₃)—CH₂—CH₂-[wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀alkyl or C₂ to C₁₀alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙ CH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., 1995, Helv. Chim. Acta 78:486-504) i.e., an alkoxyalkoxy group. A further preferred modification includes 2′-dimethylaminooxyethoxy, i.e., a O(CH₂)₂ON(CH₃)₂ group, also known as 2′-DMAOE.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition 30:613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941 and U.S. Pat. No. 5,750,692.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol (Letsinger et al., 1989, Proc. Natl. Acad. Sci. USA 86:6553-6556), cholic acid (Manoharan et al., 1994, Bioorg. Med. Chem. Let. 4:1053-1060), a thioether, e.g., hexyl-S-tritylthiol (Manoharan et al., 1992, Ann. N.Y. Acad. Sci. 660:306-309; Manoharan et al., 1993, Bioorg. Med. Chem. Let. 3:2765-2770), a thiocholesterol (Oberhauser et al., 1992, Nucl. Acids Res. 20:533-538), an aliphatic chain, e.g., dodecandiol or undecyl residues (Saison-Behmoaras et al., 1991, EMBO J. 10:1111-1118; Kabanov et al., 1990, FEBS Lett. 259:327-330; Svinarchuk et al., 1993, Biochimie 75:49-54), a phospholipid, e.g., di-hexadecyl-rac-glycerol or triethylammonium 1,2-di-O-hexadecyl-rac-glycero-3-H-phosphonate (Manoharan et al., 1995, Tetrahedron Lett. 36:3651-3654; Shea et al., 1990, Nucl. Acids Res. 18:3777-3783), a polyamine or a polyethylene glycol chain (Manoharan et al., 1995, Nucleosides & Nucleotides 14:969-973), adamantane acetic acid (Manoharan et al., 1995, Tetrahedron Lett. 36:3651-3654), a palmityl moiety (Mishra et al., 1995, Biochim. Biophys. Acta 1264:229-237), or an octadecylamine or hexylamino-carbonyl-oxycholesterol moiety (Crooke et al., 1996, J. Pharmacol. Exp. Ther. 277:923-937.

Representative U.S. patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; and 5,688,941.

It is not necessary for all positions in a given antisense compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The compounds of the invention may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative U.S. patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

The invention also provides kits for carrying out the various screening assays and methods of the invention. Such kits will typically include a yfhC polypeptide or a polynucleotide or host cell capable of expressing the same and a yfhC substrate, or a polynucleotide or host cell capable of expressing the same. The kit may further include additional components useful for carrying out the assays and methods. Non-limiting examples of such additional components include labels, labeling reagents, binding buffers, etc. The kit may also include instructions for teaching its method of uses. In one embodiment, the kit comprises as isolated yfhC polypeptide and a yfhC substrate. In another embodiment, the kit comprises a cell engineered to over express a yfhC polypeptide and an isolated yfhC substrate.

6.4.1 Uses of yfhC Polypeptide Inhibitory Compounds

As discussed previously, the antisense oligonucleotides of the invention and/or the inhibitory compounds identified by the above-described screening methods (referred to collectively as “active compounds”), can be used in a variety of in vitro, in vivo and ex vivo applications. For example, the active compounds can be used as disinfectants or preservatives to avoid bacterial growth in, or on, for example, medical equipment. They can be used as controls or standards in assays involving inhibition of yfhC polypeptides, and importantly, they can be used in therapeutic contexts to treat or prevent microbial infections.

When used to treat or prevent such diseases, the active compounds may be administered singly, as mixtures of one or more active compounds or in mixture or combination with other agents useful for treating such diseases and/or symptoms associated with such diseases. The active compounds may be administered per se or as pharmaceutical compositions.

Pharmaceutical compositions comprising the active compounds of the invention may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the active compounds into preparations which can be used pharmaceutically. The actual pharmaceutical composition administered will depend upon the mode of administration. Virtually any mode of administration may be used, including, for example topical, oral, systemic, inhalation, injection, transdermal, etc.

The active compound may be formulated in the pharmaceutical compositions per se, or in the form of a pharmaceutically acceptable salt, ester, salt of such ester or other form which, upon administration to animals and humans is capable of providing, either directly or indirectly, the active compound. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the antisense oligonucleotides of the invention are prepared as SATE [(S-acetyl-2-thioethyl)phosphate] derivatives according to the methods disclosed in International Publication No. WO 93/24510 to Gosselin et al., published Dec. 9, 1993 or in International Publication No. WO 94/26764 to Imbach et al.

As used herein, the expression “pharmaceutically acceptable salt” means those salts which retain substantially the biological effectiveness and properties of the active compound and which is not biologically or otherwise undesirable. Such salts may be prepared from inorganic and organic acids and bases, as is well-known in the art. Typically, such salts are more soluble in aqueous solutions than the corresponding free acids and bases.

For example, pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., 1977, “Pharmaceutical Salts,” J. Pharm. Sci. 66:1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

Pharmaceutically acceptable acid addition salts include organic or inorganic acid salts of the amines. Preferred acid additions salts are the hydrochlorides, acetates, salicylates, nitrates and phosphates.

Other suitable pharmaceutically acceptable salts are well known to those skilled in the art and include basic salts of a variety of inorganic and organic acids, such as, for example, hydrochloric acid, hydrobromic acid, sulfuric acid or phosphoric acid; with organic carboxylic, sulfonic, sulfo or phospho acids or N-substituted sulfamic acids, acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example, glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), ascorbic acid, etc. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For antisense oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine and iodine.

6.4.2 Pharmaceutical Compositions and Administration

For topical administration, the active compound(s) may be formulated as solutions, gels, ointments, creams, suspensions, etc. as are well-known in the art.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampoules or in multidose containers, and may contain added preservatives.

Alternatively, the injectable formulation may be provided in powder form for reconstitution with a suitable vehicle, including but not limited to, sterile pyrogen free water, buffer, dextrose solution, etc., before use. To this end, the active compound(s) may dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form, for example, of tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

For buccal administration, the compositions may take the form of tablets or lozenges formulated in conventional manner.

For rectal and vaginal routes of administration, the active compound(s) may be formulated as solutions (for retention enemas) suppositories or ointments containing conventional suppository bases such as cocoa butter or other glycerides.

For administration by inhalation, the active compound(s) can be conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

For prolonged delivery, the active compound(s) can be formulated as a depot preparation, for administration by implantation; e.g., subcutaneous, intradermal, or intramuscular injection. Thus, for example, the active ingredient may be formulated with suitable polymeric or hydrophobic materials (e.g., as an emulsion in an acceptable oil) or ion exchange resins, or as sparingly soluble derivatives; e.g., as a sparingly soluble salt.

Alternatively, transdermal delivery systems manufactured as an adhesive disc or patch which slowly releases the active compound(s) for percutaneous absorption may be used. To this end, permeation enhancers may be used to facilitate transdermal penetration of the active compound(s). Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems may be employed. Liposomes and emulsions are well-known examples of delivery vehicles that may be used to deliver active compounds(s). Certain organic solvents such as dimethylsulfoxide (DMSO) may also be employed, although usually at the cost of greater toxicity.

Compositions and formulations suitable for administering antisense oligonucleotides are described in myriad patents. A specific example is U.S. Pat. No. 6,069,008.

The pharmaceutical compositions may, if desired, be presented in a pack or dispenser device which may contain one or more unit dosage forms containing the active compound(s). The pack may, for example, comprise metal or plastic foil, such as a blister pack. The pack or dispenser device may be accompanied by instructions for administration.

6.4.3 Effective Dosages

The active compound(s) of the invention, or compositions thereof, are generally used in an amount effective to treat or prevent an infection in the subject being treated. The compound(s) may be administered therapeutically to a subject suffering from an infection to achieve a therapeutic benefit or prophylactically to a patient at risk of developing an infection to achieve a prophylactic benefit. By therapeutic benefit is meant eradication or amelioration of the infection and/or eradication or amelioration of one or more of the symptoms associated with the infection such that the patient reports an improvement in feeling or condition, notwithstanding that the patient may still be infected. For example, administration of an active compound to a patient suffering from an infection provides therapeutic benefit not only when the underlying infection is eradicated or ameliorated, but also when the patient reports a decrease in the severity or duration of the symptoms associated with the infection. Therapeutic benefit also includes halting or slowing the progression of the infection, regardless of whether the infection is eradicated.

For prophylactic administration, the active compound may be administered to a patient at risk of developing an infection. For example, the active compound(s) may be administered to patients in certain clinical settings to prevent or avoid the incidence of nosoconial infections, particularly those caused by strains of bacterial resistant to conventional antibiotics.

The amount of active compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Initial dosages may be estimated initially from in vitro assays. For example, an initial dosage for use in animals may be formulated to achieve a circulating blood or serum concentration of active compound that is at or above the IC₅₀ of the particular active compound against the particular pathogen. Calculating dosages to achieve such circulating blood or serum concentrations taking into account the bioavailability of the particular active compound is well within the capabilities of skilled artisans. For guidance, the reader is referred to Fingl & Woodbury, “General Principles,” In: The Pharmaceutical Basis of Therapeutics, Chapter 1, pp. 1-46, 1975, and the references cited therein.

Initial dosages can also be estimated from in vivo data, such as animal models. Animals models useful for testing the efficacy of compounds to treat or prevent infections characterized are well-known in the art.

Dosage amounts will typically be in the range of from about 100 mg/day to about 1 g/day, or in the range of about 2-20 mg/kg/day, but may be higher or lower, depending upon, among other factors, the activity of the active compound, its bioavailability, the mode of administration and various factors discussed above. Dosage amount and interval may be adjusted individually to provide plasma levels of the active compound(s) which are sufficient to maintain therapeutic or prophylactic effect. In cases of local administration or selective uptake, such as local topical administration, the effective local concentration of active compound(s) may not be related to plasma concentration. Skilled artisans will be able to optimize effective local dosages without undue experimentation.

The compound(s) may be administered once per day, a few or several times per day, or even multiple times per day, depending upon, among other things, the indication being treated and the judgment of the prescribing physician.

Preferably, the active compound(s) will provide therapeutic or prophylactic benefit without causing substantial toxicity. Toxicity of the active compound(s) may be determined using standard pharmaceutical procedures. The dose ratio between toxic and therapeutic (or prophylactic) effect is the therapeutic index. Active compound(s) that exhibit high therapeutic indices are preferred.

The invention having been described, the following examples are offered by way of illustration and not limitation.

### 7. EXAMPLES

7.1 E. coli yfhC Polypeptide Catalyzes Deamination of a Truncated Substrate

This example demonstrates that an E. coli yfhC polypeptide having a sequence corresponding to FIG. 1B recognizes truncated tRNA^{Arg(ACG)} substrates. In particular, this example demonstrates that an RNA hairpin (SEQ. ID NO:1) corresponding to the anticodon stem loop of E. coli tRNA^{Arg2} acts as a substrate for this yfhC polypeptide.

7.1.1 Experimental Protocol

The experimental protocol consisted of two steps. First an in vitro reaction was conducted using a defined yfhC substrate and the yfhC polypeptide supplied as a lysate from an over-producing strain of bacteria. The reactions were then digested enzymatically to mononucleotides with a mixture of three enzymes (nuclease P1, snake venom phosphodiesterase and alkaline phosphatase and the resultant digest analyzed by HPLC to monitor for the appearance of inosine (the product of the yfhC polypeptide deamination reaction).

It has been previously demonstrated that the yfhC polypeptide over-expression lysates deaminate E. coli tRNA^{Arg2(ACG)} to yield tRNA^{Arg(ICG)}. In this experiment, a series of reactions were run with a synthetic RNA corresponding to the following anticodon stem loop 17-mer of E. coli tRNA^{Arg2}, in both the linear and folded form (the anticodon is underlined):

| | |
|---|---|
| C U C G G C U ACG A A C G A G | (SEQ ID NO:1) |

All reactions were carried out with the indicated amounts of yfhC substrate and yfhC polypeptide (in the form of a cell lysate) in a final volume of 50 μl. For experiments employing refolded yfhC substrate, the substrate was first boiled and slowly cooled in refolding buffer (30 mM HEPES, pH 7.9, 150 mM NaCl, 10 mM Mg Cl₂) prior to adding to the reaction mixture. All reactions were run for 30 min. prior to quenching (by boiling for 2 min. at 100° C.), except for control reactions #9 and 11, which were quenched immediately. Following quenching, all reactions were stored overnight at −80° C., extracted with phenol (1X) and chloroform (2X) to remove, the proteins, and the nucleic acids precipitated with ethanol. The pellets were resuspended in 50 μl H₂O, digested with a mixture of nuclease P1, snake venom phosphodiesterase 1 and alkaline phosphatase (all enzymes in excess) and analyzed by HPLC (Develosil C-30 column, 2 mm×250 mm; Nomura Chemical, Japan). Elutions were carried out using an approximately linear gradient over 20 min. followed by an approximately exponential gradient from 20-25 min. of Buffer B (40% acetonitrile in water) in Buffer A (5 mM ammonium acetate, pH 5.3) using a flow rate of 300 μl/min. Two controls negative for the yfhC polypeptide (reactions 4 and 7) utilized lysates of cells transformed with a non-yfhC expressing vector derived from the pBAD plasmid (Invitrogen, San Diego, Calif.). The various reactions were carried out:
Reaction 4: Negative Control
   2 μg pBAD
   20 μg stem loop
   30 min.
Reaction 7: Negative Control
   2 μg pBAD
   8 μg tRNA^{Arg2}
   30 min.
Reaction 9: Negative Control
   2 μg yfhC polypeptide
   20 μg stem loop
   Quenched immediately
Reaction 10: Refolded yfhC substrate
   2 μg yfhC polypeptide
   20 μg refolded stem loop
   30 min.
Reaction 11: yfhC substrate
   2 μg yfhC polypeptide
   20 μg stem loop
   30 min.
Reaction 12: Negative Control
   2 μg yfhC polypeptide
   8 μg tRNA^{Arg2}
   Quenched immediately
Reaction 13: Positive Control
   2 μg yfhC polypeptide
   8 μg tRNA^{Arg2}
   30 min.

7.1.2 Results

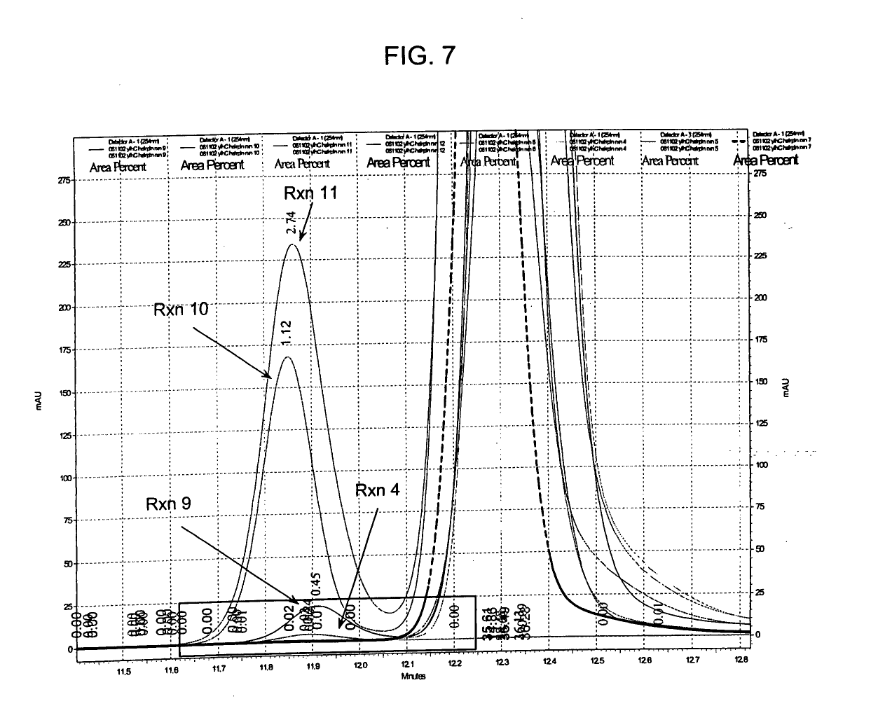

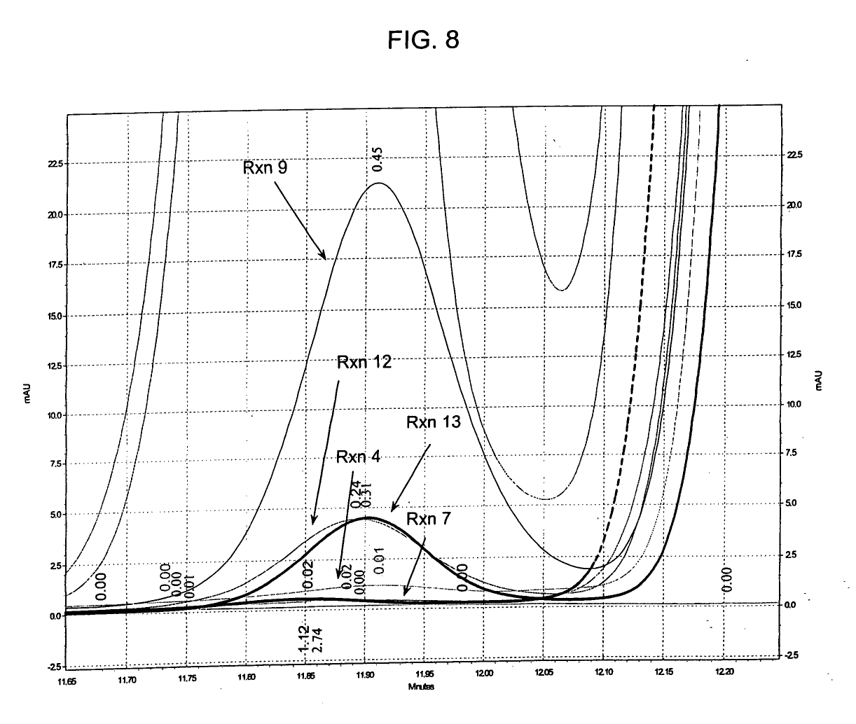

The inosine region of the HPLC elution profiles of the various reactions are provided in FIG. 7. In the elution profile, the inosine peak (approx. 11.7-12.1 min.) is labeled. A detailed blow-up of this inosine peak is provided in FIG. 8. As evident from FIGS. 7 and 8, negative control reactions 4 and 7 show minimal activity. As these controls did not over express the yfhC polypeptide, the minimal activity observed was likely due to residual inosine carried over from the lysate or residual wild-type yfhC polypeptide activity, carried over from the lysate. Control reactions 12 and 13, which included the full length E. Coli tRNA^{Arg2} substrate, showed moderate activity. The stem loop, both boiled and unboiled to refold, showed the greatest-activity, confirming this RNA as a substrate for the yfhC polypeptide.

All references cited throughout the application are incorporated herein by reference in their entireties for all purposes.

1. A method of identifying a compound that inhibits a yfhC polypeptide, comprising the steps of contacting a yfhC polypeptide and a yfhC substrate with a candidate compound of interest and detecting the presence or absence of a modified yfhC substrate, where the absence of the modified substrate or a reduction in the amount of the modified substrate produced as compared to a control reaction identifies the candidate compound as an inhibitor of the yfhC polypeptide.

2. The method of claim 1 in which the yfhC polypeptide is a bacterial yfhC polypeptide.

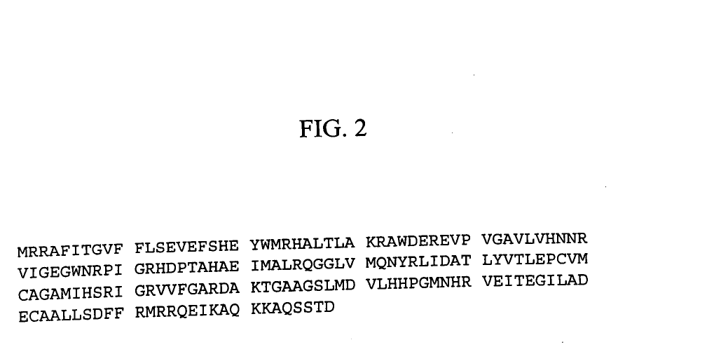

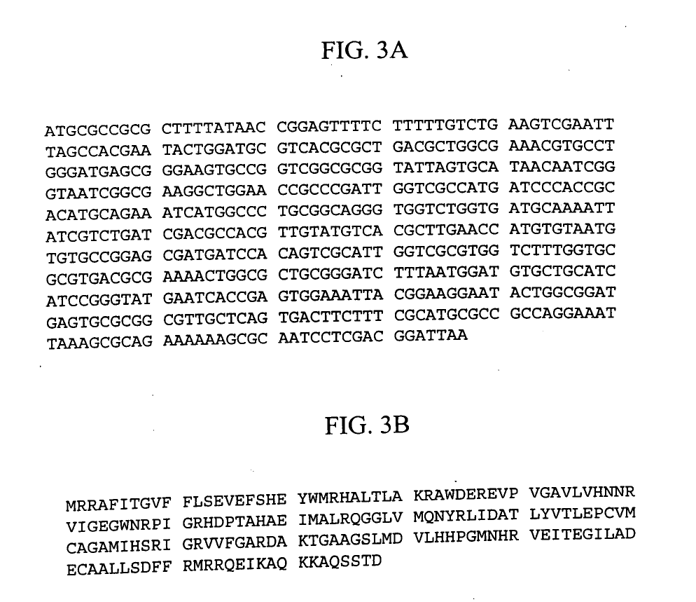

3. The method of claim 2 in which the bacterial yhfC polypeptide comprises an amino acid sequence corresponding to the polypeptide of FIG. 1A, FIG. 2, FIG. 3B or any of the accession nos. of TABLE 1.

4. The method of claim 1 in which the yfhC substrate is a polynucleotide comprising from 3 to about 80 nucleotides that includes an ACG loop.

5. The method of claim 4 in which the polynucleotide is an RNA.

6. The method of claim 4 in which the polynucleotide comprises from 7 to about 21 nucleotides and is capable of forming a hairpin secondary structure, and wherein said ACG sequence resides in a loop region of the hairpin.

7. The method of claim 4 in which the yfhC substrate is a bacterial tRNA^{Arg(ACG)}.

8. The method of claim 7 in which the tRNA^{Arg(ACG)} is selected from the group of tRNAs depicted in FIG. 5.

9. The method of claim 4 in which the yfhC substrate is an RNA comprising from 7 to 17 nucleotides that has a nucleotide sequence derived from the anticodon stem loop of a bacterial tRNA^{Arg(ACG)}.

10. The method of claim 9 in which the bacterial tRNA^{Arg(ACG)} is selected from the group of tRNAs depicted in FIG. 5.

11. The method of claim 9 in which the yfhC substrate is selected from the group consisting of

| | |
|---|---|
| C U/T C G G C U/T ACG A A C C G A G; | (SEQ ID NO:1) |
| U/T C G G C U/T ACG A A C C G A; | (SEQ ID NO:2) |
| C G G C U/T ACG A A C C G; | (SEQ ID NO:3) |
| G G C U/T ACG A A C C | (SEQ ID NO:4) |
| and | |
| G C U/T ACG A A C. | (SEQ ID NO:5) |

12. A method of identifying an antibacterial compound, comprising the steps of contacting a yfhC polypeptide and a yfhC substrate with a candidate compound of interest and detecting the presence or absence of a modified yfhC substrate, where the absence of the modified substrate or a reduction in the amount of the modified substrate produced as compared to a control reaction identifies the candidate compound as an antibacterial compound.

13. An antisense oligonucleotide composed of from 8 to about 30 nucleotides and which includes a sequence which hybridizes to a portion of a polynucleotide encoding a bacterial yfhC polypeptide.

14. A compound identified by the screening method of claim 1 or claim 12.

15. A pharmaceutical composition comprising a compound that inhibits a yfhC polypeptide or the expression of a yhfC polypeptide and a pharmaceutically acceptable carrier, excipient or diluent.

16. The composition of claim 15 in which the compound is an antisense oligonucleotide according to claim 13.

17. The composition of claim 16 in which the compound is a compound identified by the screening method of claim 1 or claim 12.

18. A method of inhibiting the growth or replication of a bacterium, comprising administering to the bacterium an amount of a compound that inhibits a yfhC polypeptide effective to inhibit its growth or replication.

19. The method of claim 18 in which the compound is an antisense oligonucleotide according to claim 13.

20. The method of claim 18 in which the compound is a compound identified by the screening method of claim 1 or claim 12.

21. A method of treating or preventing a bacterial infection in a subject, comprising administering to a subject in need thereof an amount of a compound that inhibits a yfhC polypeptide effective to treat or prevent the infection.

22. The method of claim 21 in which the compound is an antisense oligonucleotide according to claim 13.

23. The method of claim 21 in which the compound is a compound identified by the screening method of claim 1 or claim 12.

24. A kit comprising a yfhC polypeptide and a yfhC substrate.
